(19) United States
(12) Patent Application Publication
(10) Pub. No.: US 2006/0058636 A1
(43) Pub. Date: Mar. 16, 2006

(54) Method for tracking the movement of a particle through a geometric model for use in radiotherapy

(76) Inventors: Charles A. Wemple, Idaho Falls, ID (US); Daniel E. Wessol, Bozeman, MT (US)

Correspondence Address: Alan D. Kirsch;BBWI, PO BOX 1625, IDAHO FALLS, ID, 83415-3899, US

(21) Appl. No.: 10/940,349

(22) Filed: Sep. 13, 2004

(51) Int. Cl. A61B 5/05

(52) U.S. Cl. 600/411

(57) ABSTRACT

A method for tracking the movement of a particle through a geometric model so as to facilitate the development of a dosimetry plan includes providing a particle with a short mean free path length; providing a geometric model which has a boundary which separates regions of the geometric model having different densities and/or compositions; arranging a first plurality of substantially uniform volume elements having a predetermined size into the geometric model; and arranging a second plurality of substantially uniform volume elements having a predetermined size less than that of the first plurality of substantially uniform volume elements, and in overlaying relation relative to the first plurality of substantially uniform volume elements.

### GOVERNMENT RIGHTS

The United States Government has rights in the following invention pursuant to Contract No. DE-AC07-99ID13727 between the U.S. Department of Energy and Bechtel BWXT Idaho, LLC.

### TECHNICAL FIELD

The present invention relates to a method for tracking the movement of particles through a geometric model so as to facilitate the development of a dosimetry plan, and more specifically to a methodology which includes the step of traversing a particle along the particle track, and across the boundary which separates regions of the geometric model which have different densities and/or compositions, while minimizing the use of a floating point calculation to determine the particle location at the boundary crossing.

### BACKGROUND OF THE INVENTION

The present invention relates generally to radiation therapy and more specifically to the analytical computations for the dosimetric planning thereof. In this regard, radiation transport calculations for radiotherapy applications have traditionally used Monte Carlo methods because of the highly complex geometry involved, and the inherent multiple particle nature of the calculations. In this regard, the use of medical image sets to define the calculation geometry allowed for more exact descriptions of tissues and organs in the patient. Most radiotherapy treatment planning systems define the organ/tissues by outlining the regions using some variant of a spline to define the region boundary. This requires the use of floating-point arithmetic to perform the particle tracking function.

In U.S. Pat. No. 6,175,761 the inventors disclosed a method that used medical images to define an array of uniform volume elements (univels), which are identical rectangular parallelepipeds, to model the patients' geometry. This methodology allowed the particle tracking functions to be formed using integer arithmetic, with the floating-point computations only required for the final location of the particle at a boundary crossing. This methodology greatly reduced the computation time involved in the tracking functions, by removing the need for expensive floating-point arithmetic at each stage of the particle tracking procedure.

While the methodology described in U.S. Pat. No. 6,175,761 has operated with a great deal of success, several shortcomings have been identified and which have detracted from its usefulness. For example, it has been recognized that the univel method of computation only achieved efficient operation when the average distance between the particle collisions, or mean free path was large relative to the size of the univel. This methodology therefore was ideal for neutron transport but was viewed as not any more efficient than conventional methodologies for the coupled photon-electron transport.

Therefore an improved method for tracking the movement of a particle through a geometric model so as to facilitate the development of a dosimetry plan, and which addresses the shortcomings attendant with the prior art methodology and practices utilized heretofore is the subject matter of the present application.

### SUMMARY OF THE INVENTION

Therefore one aspect of the present invention relates to a method for tracking the movement of a particle through a geometric model so as to facilitate the development of a dosimetry plan, and which includes providing a particle with a short mean free path length; providing a geometric model which has a boundary which separates regions of the geometric model having different densities and/or compositions; arranging a first plurality of substantially uniform volume elements having a predetermined size into the geometric model; and arranging a second plurality of substantially uniform volume elements having a predetermined size less than that of the first plurality of substantially uniform volume elements, and in overlaying relation relative to the first plurality of substantially uniform volume elements.

Another aspect of the present invention relates to a method for rapidly tracking the movement of a particle through a geometric model so as to facilitate the development of a dosimetry plan, and which includes providing a geometric model which includes regions having different densities and/or compositions, and wherein a boundary is defined between the regions having the different densities and/or compositions; arranging a first plurality of substantially uniform volume elements into the geometric model; creating a particle with a short mean free path length in at least one of the first plurality of substantially uniform volume elements which is located in at least one of the regions; arranging a second plurality of substantially uniform volume elements which overlays at least one of the first plurality of substantially uniform volume elements where the particle having the short mean free path length is created, and wherein the second plurality of substantially uniform volume elements have a size which is small in relative comparison to the short mean free path length of the particle; describing the movement of the particle through the geometric model with a particle track which has a primary direction of movement; and traversing the particle having the short mean free path length along the particle track, and across the boundary which separates the regions having the different densities and/or compositions, by minimizing the use of a floating point computation, to determine the particle location at the boundary crossing.

Yet further, another aspect of the method for tracking the movement of a particle through a geometric model so as to facilitate the development of a dosimetry plan, which includes obtaining a medical image of a treatment volume and which includes a plurality of pixels of information; providing a particle with a short mean free path length; providing a geometric model which defines regions in the treatment volume having different densities and/or compositions, and wherein a boundary is defined between the regions having the different densities and/or compositions; converting the pixels of information derived from the treatment volume into a first plurality of substantially uniform volume elements having a predetermined size; arranging the first plurality of substantially uniform volume elements having predetermined dimensions into the geometric model which defines the regions having different densities and/or compositions, and wherein the particle having the short mean free path length is created in at least one of the first plurality of substantially uniform volume elements which is located in at least one of the regions; arranging a second plurality of substantially uniform volume elements which overlays at least one of the first plurality of uniform volume elements where the particle having the short mean free path length is created, and wherein the second plurality of uniform volume elements have a predetermined size which are less than the predetermined size of the first plurality of uniform volume elements, and are further small in size in relative comparison to the mean free path length of the particle; describing the movement of the particle in integer base increments through the geometric model with a particle track which has a primary direction of movement; traversing the particle along the particle track, and across the boundary which separates the regions of the geometric model which have different densities and/or compositions, while minimizing the use of a floating point calculation to determine the particle location at the boundary crossing; and calculating a dosimetry plan for conducting radiotherapy for an area of the treatment volume which is in juxtaposed relation relative to the boundary which separates the regions having the different densities and/or compositions by utilizing the particle location at the boundary location.

These and other aspects of the present invention will be described in greater detail hereinafter.

### BRIEF DESCRIPTION OF THE DRAWINGS

Preferred embodiments of the invention are described below with reference to the following accompanying drawings.

FIG. 1 is an exemplary diagram showing a medical image of a treatment volume and which includes both a first plurality of uniform volume elements having a predetermined size, and a second plurality of uniform volume elements having a size which is less than the first size.

FIG. 2 is an exemplary diagram for understanding the conversion of pixels of medical imagery into a geometric model and for mapping the pixels into an array in accordance with the teachings of the present invention.

### DETAILED DESCRIPTION OF THE PREFERRED EMBODIMENTS

This disclosure of the invention is submitted in furtherance of the constitutional purposes of the U.S. Patent Laws “to promote the progress of science and useful arts” (Article 1, Section 8).

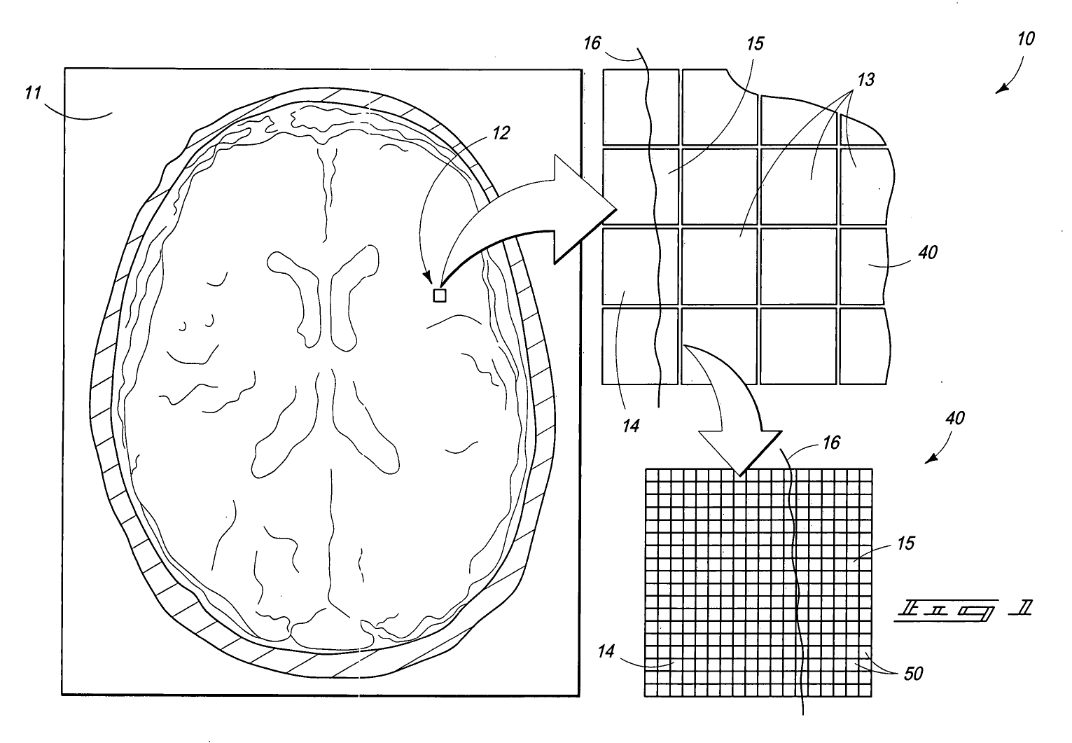

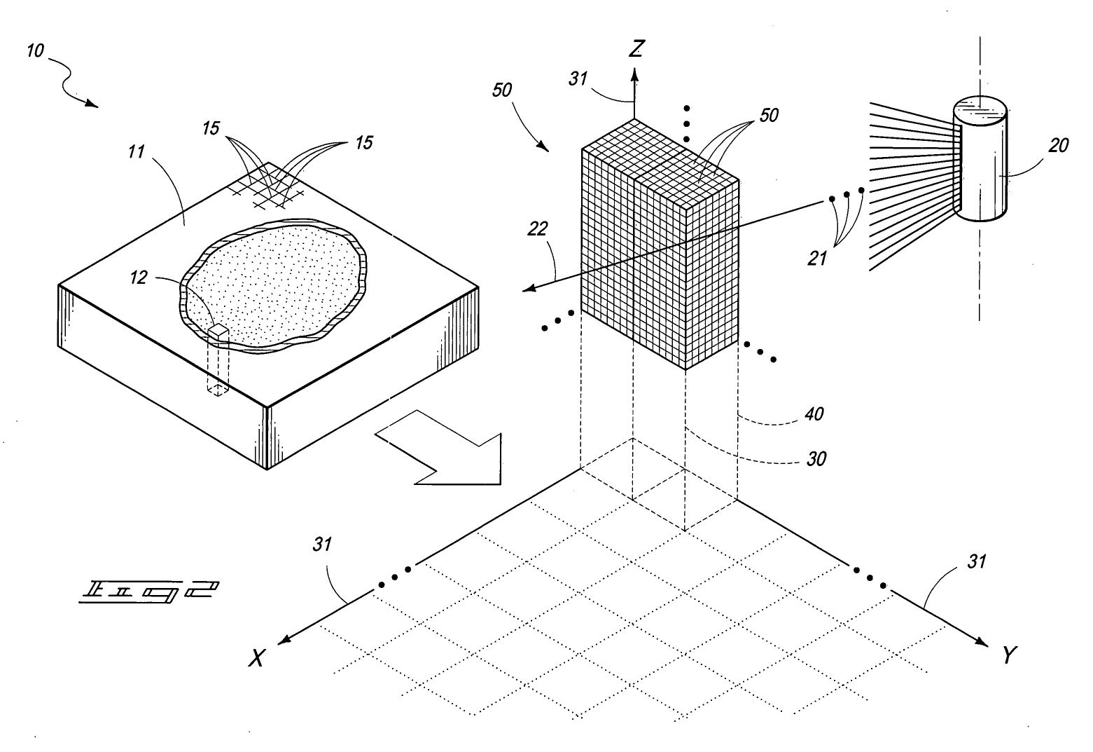

The method for tracking the movement of a particle through a geometric model so as to facilitate the development of a dosimetry plan is best indicated by the numeral 10 in FIGS. 1 and 2. In the methodology of the present invention, the first step of the present method includes obtaining a medical image 11 of a treatment volume 12 and which includes a plurality of pixels of information 13. The treatment volume 12 as seen in FIG. 1 includes a plurality of first and second regions 14 and 15. The first region having a first density, and the second region having a second density different from the first density. A border 16 is defined between the first and second region. Referring now to FIG. 2, another step in the method of the present invention is to provide a particle with a short mean free path length. In this regard, a particle generator 20 is generally shown in FIG. 2 and which produces a plurality of particles having the short mean free path length 21. Each of the particles have a particle track 22 which is directed towards and which passes through a geometric model which is generally indicated by the numeral 30. In this regard, another step in the method for tracking the movement of a particle includes providing a geometric model 30 which defines regions in the treatment volume 15 and 16 having different densities and/or compositions. As noted above, each of these regions includes a boundary 16 defined between the regions and which has different densities and/or compositions. In the method of the present invention, the methodology includes still another step of converting the pixels of information 13 derived from the treatment volume 12 into a first plurality of substantially uniform volume elements 40 having a predetermined size. Following the conversion of the pixels into the first plurality of substantially uniform volume elements 40, the method includes another step of arranging the first plurality of substantially uniform volume elements 40 having a predetermined dimension into the geometric model 30 which defines the regions having different densities and/or compositions. In the arrangement as shown, the particle 21 having the short mean free path length is created in at least one of the first plurality of substantially uniform volume elements which is located in at least one of the regions 15 or 16.

After the step of arranging the first plurality of substantially uniform volume elements, as referenced in the paragraph above, the methodology includes another step of arranging a second plurality of substantially uniform volume elements 50 as seen in FIGS. 1 and 2 and which overlays at least one of the plurality of uniform volume elements 40 where the particle 20 having the short mean free path length is created. The second plurality of uniform volume elements 50 have a predetermined size which are less than the predetermined size of the first plurality of uniform volume elements and are further small in size in relative comparison to the short mean free path length of the particle 20. This is most clearly seen by references to FIG. 2. After the step of arranging the second plurality of substantially uniform volume elements in overlaying relation relative to the first plurality of uniform volume elements 40, the method further includes the step of describing the movement of the particle 21 in integer based increments through the geometric model 30 with a particle track 22 which has a primary direction of movement as seen in FIG. 2. This description of the movement of the particle in integer based increments through the geometric model 30 is described in significant detail in U.S. Pat. No. 6,175,761. The teachings of this patent are incorporated by reference herein. In view of the description of the integer based arithmetic that is utilized, and the computer arrangement which is employed to implement such a model, a further discussion regarding the mathematical computations and the means for accomplishing same are not warranted in this application. After the step of describing the movement of the particle in integer based increments through the geometric model 30, the method of the present invention includes a step of traversing the particle 21 across the boundary 16 which separates the regions of the geometric model 30 which have different densities and/or compositions, while minimizing the use of a floating-point calculation to determine the particle location at the boundary crossing. After the step of traversing the particle along the particle track, the method further includes the step of calculating a dosimetry plan for conducting radiotherapy for an area of the treatment volume 12 which is juxtaposed relative to the boundary 16 which separates the regions 15 and 16 having the different densities and/or compositions by utilizing the particle location at the boundary location 16.

In the methodology of the present invention, the predetermined size of the second plurality of substantially uniform volume elements 50 is determined as the product of the ratio of the densities of the least dense region of all the regions in the geometric model 30, and the region of the geometric model in which the particle having the short mean free path length 21 was created, and the predetermined size of the individual first plurality of substantially uniform volume elements 40. Still further, in connection with the present methodology, and after the step of arranging the first plurality of substantially uniform volume elements 40, the methodology further includes the step of defining a material to be associated with each of the substantially uniform volume elements.

Therefore, the method 10 for tracking the movement of a particle through a geometric model 30 so as to develop a dosimetry plan includes, in its broadest aspect, providing a particle 21 with a short mean free path length; providing a geometric model 30 which has a boundary 16 (FIG. 1) which separates regions 14 and 15 of the geometric model, and which have different densities and/or compositions; arranging a first plurality of substantially uniform volume elements 40 having a predetermined size into the geometric model; and arranging a second plurality of substantially uniform volume elements 50 having a predetermined size less than the first plurality of substantially uniform volume elements, and in overlaying relation relative to the first plurality of substantially uniform volume elements. In the methodology as described, the present arrangement includes a step of minimizing and/or eliminating the use of a floating-point computation to determine the particle 21 location at the boundary crossing 16 of the particle track 22. This methodology further includes utilizing integer based increments when traversing the particle along the particle track 22 and across the boundary, and wherein the particle track has a primary direction of movement and wherein the integer based increments are applied along the primary direction of movement.

Therefore it will be seen that the present method provides a convenient means whereby an accurate dosimetry plan can be developed for a treatment volume in a manner not possible heretofore, and by utilizing integer based arithmetic and minimizing the use of floating-point calculations to determine a particle location at a boundary crossing.

In compliance with the statute, the invention has been described in language more or less specific as to structural and methodical features. It is to be understood, however, that the invention is not limited to the specific features shown and described, since the means herein disclosed comprise preferred forms of putting the invention into effect. The invention is, therefore, claimed in any of its forms or modifications within the proper scope of the appended claims appropriately interpreted in accordance with the doctrine of equivalents.

1. A method for tracking the movement of a particle through a geometric model so as to facilitate the development of a dosimetry plan, comprising:
providing a particle with a short mean free path length;
providing a geometric model which has a boundary which separates regions of the geometric model having different densities and/or compositions;
arranging a first plurality of substantially uniform volume elements having a predetermined size into the geometric model; and
arranging a second plurality of substantially uniform volume elements having a predetermined size less than that of the first plurality of substantially uniform volume elements, and in overlaying relation relative to the first plurality of substantially uniform volume elements.

2. A method for tracking the movement of a particle as claimed in claim 1, and wherein the second plurality of substantially uniform volume elements are small in size in relative comparison to the mean short mean free path length of the particle.

3. A method for tracking the movement of a particle as claimed in claim 2, and further comprising:
after the step of arranging the second plurality of substantially uniform volume elements, describing the movement of the particle through the geometric model with a particle track which crosses the boundary separating the regions of the geometric model having different densities and/or compositions.

4. A method for tracking the movement of a particle as claimed in claim 3, and further comprising:
after the step of describing the movement of the particle through the geometric model with a particle track, traversing the particle along the particle track and across the boundary.

5. A method for tracking the movement of a particle as claimed in claim 4, and wherein the step of traversing the particle along the particle track further comprises:
minimizing and/or eliminating the use of a floating point computation to determine the particle location at the boundary crossing of the particle track.

6. A method for tracking the movement of a particle as claimed in claim 5, and wherein the step of minimizing and/or eliminating the use of a floating point computation further comprises:
utilizing integer based increments when traversing the particle along the particle track, and across the boundary, and wherein the particle track has a primary direction of movement, and wherein integer based increments are applied along the primary direction of movement.

7. A method for tracking the movement of a particle as claimed in claim 1, and wherein the step of providing the particle with the short mean free path length further includes creating the particle at one of the first plurality of substantially uniform volume elements, and wherein the step of arranging the second plurality of uniform volume elements further comprises:
overlaying the second plurality of uniform volume elements over at least one of the first plurality of substantially uniform volume elements where the particle having the short mean free path length is created.

8. A method for tracking the movement of a particle as claimed in claim 7, and wherein the predetermined size of the second plurality of substantially uniform volume elements is determined as the product of the ratio of the densities of the least dense region of geometric model, and the region of the geometric model which contains the substantially uniform volume element in which the particle having the short mean free path length was created, and the predetermined size of the individual first plurality of substantially uniform volume elements.

9. A method for rapidly tracking the movement of a particle through a geometric model so as to facilitate the development of a dosimetry plan, comprising:
providing a geometric model which includes regions having different densities and/or compositions, and wherein a boundary is defined between the regions having the different densities and/or compositions;
arranging a first plurality of substantially uniform volume elements into the geometric model;
creating a particle with a short mean free path length in at least one of the first plurality of substantially uniform volume elements which is located in at least one of the regions;
arranging a second plurality of substantially uniform volume elements which overlays at least one of the first plurality of substantially uniform volume elements where the particle having the short mean free path length is created, and wherein the second plurality of substantially uniform volume elements have a size which is small in relative comparison to the short mean free path length of the particle;
describing the movement of the particle through the geometric model with a particle track which has a primary direction of movement; and
traversing the particle having the short mean free path length along the particle track, and across the boundary which separates the regions having the different densities and/or compositions, by minimizing the use of a floating point computation, to determine the particle location at the boundary crossing.

10. A method for tracking the movement of a particle as claimed in claim 9, and wherein each of the first and second plurality of substantially uniform volume elements have a predetermined size, and wherein the predetermined size of the second plurality of substantially uniform volume elements is less than the predetermined size of the first plurality of substantially uniform volume elements.

11. A method for tracking the movement of a particle as claimed in claim 10, and wherein the step of describing the movement of the particle through the geometric model is performed in integer based increments.

12. A method for tracking the movement of a particle as claimed in claim 10, and wherein the predetermined size of the second plurality of substantially uniform elements is determined as the product of the ratio of the densities of the least dense region of the geometric model, and the region in which the particle having the short mean free path length was created, and the predetermined size of the individual first plurality of the substantially uniform elements.

13. A method for tracking the movement of a particle as claimed in claim 9, and further comprising:
after the step of traversing the particle having the short mean free path length along the particle track, calculating the dosimetry plan for conducting radiotherapy for an area located in juxtaposed relation relative to the boundary which separates the regions having different densities and/or compositions.

14. A method for tracking the movement of a particle as claimed in claim 13, and further comprising:
before the step of arranging a first plurality of substantially uniform volume elements into the geometric model, obtaining a medical image of a treatment volume, and which includes a plurality of pixels of information; and
converting the pixels of information derived from the treatment volume into the first plurality of substantially uniform volume elements.

15. A method for tracking a movement of a particle through a geometric model so as to facilitate the development of a dosimetry plan, comprising:
obtaining a medical image of a treatment volume and which includes a plurality of pixels of information;
providing a particle with a short mean free path length;
providing a geometric model which defines regions in the treatment volume having different densities and/or compositions, and wherein a boundary is defined between the regions having the different densities and/or compositions;
converting the pixels of information derived from the treatment volume into a first plurality of substantially uniform volume elements having a predetermined size;
arranging the first plurality of substantially uniform volume elements having predetermined dimensions into the geometric model which defines the regions having different densities and/or compositions, and wherein the particle having the short mean free path length is created in at least one of the first plurality of substantially uniform volume elements which is located in at least one of the regions;
arranging a second plurality of substantially uniform volume elements which overlays at least one of the first plurality of uniform volume elements where the particle having the short mean free path length is created, and wherein the second plurality of uniform volume elements have a predetermined size which are less than the predetermined size of the first plurality of uniform volume elements, and are further small in size in relative comparison to the mean free path length of the particle;
describing the movement of the particle in integer base increments through the geometric model with a particle track which has a primary direction of movement;
traversing the particle along the particle track, and across the boundary which separates the regions of the geometric model which have different densities and/or compositions, while minimizing the use of a floating point calculation to determine the particle location at the boundary crossing; and
calculating a dosimetry plan for conducting radiotherapy for an area of the treatment volume which is in juxtaposed relation relative to the boundary which separates the regions having the different densities and/or compositions by utilizing the particle location at the boundary location.

16. A method for tracking a movement of a particle, as claimed in claim 15, and wherein the predetermined size of the second plurality of substantially uniform elements is determined as the product of the ratio of the densities of the least dense region of the geometric model, and the region of the geometric model in which the particle having the short mean free path length was created, and the predetermined size of the individual first plurality of the substantially uniform elements.

17. A method for tracking a movement of a particle, as claimed in claim 16, and further comprising:
after the step of arranging the first plurality of substantially uniform elements, defining a material to be associated with each of the substantially uniform volume elements.
